(19) United States
(12) Patent Application Publication
(10) Pub. No.: US 2016/0331867 A1
(43) Pub. Date: Nov. 17, 2016

(54) MULTILAYERED RETINAL CELL IMPLANT

(71) Applicant: Taipei Veterans General Hospital, Taipei City (TW)

(72) Inventor: Shih-Hwa Chiou, Taipei City (TW)

(21) Appl. No.: 15/154,649

(22) Filed: May 13, 2016

Related U.S. Application Data
(60) Provisional application No. 62/160,799, filed on May 13, 2015.

(51) Int. Cl. A61L 27/36; A61L 27/34; A61L 27/18

(52) U.S. Cl. CPC A61L 27/3604; A61L 27/18; A61L 27/34; A61L 27/3641; A61L 2430/16

(57) ABSTRACT

The present invention relates to a method for preparing a multilayered retinal cell implant. The method comprises coating a substrate with laminin to obtain a laminin modified substrate and growing retinal cells derived from stem cells or induced pluripotent stem cells (iPSCs) on the laminin modified substrate, wherein the retinal cells as grown include multiple layers of retinal cells, and provides properties and efficacy facilitating retinal repair.

### RELATED APPLICATION

The present application claims priority under 35 U.S.C. §119(e) to U.S. Provisional Patent Application No. 62/160,799, filed May 13, 2015, the content of which is herein incorporated by reference in its entirety.

### FIELD OF THE INVENTION

The present invention generally relates to a multiplayered retinal cell impplant for repairing a retinal defect or disorder.

### BACKGROUND OF THE INVENTION

Age-related macular degeneration (AMD) is a worldwide leading cause of blindness especially in developing countries. Patients with end-stage AMD lost their central vision permanently mainly due to fibrovascular scar or atrophy of retinal pigment epithelium (RPE) and photoreceptors in macula. Current treatments focused on controlling growth and leakage of choroidal neovessels in wet-type AMD by injecting anti-vascular endothelial growth factor (VEGF) repeatedly. (Martin et al. Ranibizumab and bevacizumab for neovascular age-related macular degeneration. N Engl J Med. 2011; 364:1897-908.) The visual outcomes were usually restricted due to persistence of the fibrous tissue and the loss of RPE and photoreceptors. To prevent the formation and the advanced destruction from neovessels in wet-type AMD, many new therapies were proposed, including inhibition of platelet-derived growth factor, neutralization of the sphingosine-l-phosphate, anti-integrin oligopeptide, radiation therapy, surgical implant and gene therapy. (Pecen & Kaiser. Current phase ½ research for neovascular age-related macular degeneration. Current opinion in ophthalmology. 2015; 26:188-93.) Besides, a humanized monoclonal antibody targeting complement factor D, lampalizumab, was also designed for treating the geographic atrophy in dry-type AMD (Do DV et al. A phase is dose-escalation study of the anti-factor D monoclonal antibody fragment FCFD4514S in patients with geographic atrophy. Retina (Philadelphia, Pa). 2014; 34:313-20.) However, scarce treatments focused their effect on RPE and neurosensory retina, of which the dysfunction and degeneration may weaken the blood-retina-barrier and cause AMD originally. For this, stem cell therapy provides another ideal opportunity to treat the retinal-degenerative diseases from roots.

Given the shortage of therapeutic drugs to treat the advanced AMD, transplantation of RPE, photoreceptor, or other retinal cells is an alternative way to repair the damaged retina in AMD patients. However, obtaining a sufficient number of suitable donor RPE and photoreceptors for ex vivo transplantation in rescuing the visual dysfunction of AMD is still an obstacle for such therapy. Therefore, pluripotent stem cell-based therapy, such as embryonic stem cells (ESC) and induced pluripotent cells (iPSC), is a potential resolution for the limited donor's RPE in regeneration medicine. (Can et al. Development of human embryonic stem cell therapies for age-related macular degeneration. Trends in neurosciences. 2013; 36:385-95.) It was reported that the polarized monolayer of RPE showed better survival and growth compared with suspended RPE cells. (Diniz et al. Subretinal implantation of retinal pigment epithelial cells derived from human embryonic stem cells: improved survival when implanted as a monolayer. Investigative ophthalmology & visual science. 2013; 54:5087-96.) Transplanting pluripotent stem cell-differentiated RPE as a sheet of monolayer has more potential for a successful retinal repair, particularly for the geographic atrophy in dry-AMD patients that need to repair a rather large area of retina. (Reardon & Cyranoski. Japan stem-cell trial stirs envy. Nature. 2014; 513:287-8.) However, the biosafety and efficacy of the transplantable materials, as well as the visual-functional improvement of the implanted RPE cells in the subretinal space have not been confirmed.

Accordingly, it is desirable to develop a new scaffolding cell graft for repairing retinal defects, particularly age-related macular degeneration (AMD).

### SUMMARY OF THE INVENTION

Accordingly, the invention provides a method for preparing a multilayered retinal cell implant and the products prepared therefrom, characterized by the growth of retinal cells on a laminin modified substrate. The multilayered retinal cell implant as obtained contains multiple layers of various retinal cells, including at least retinal pigment epithelium cells (RPEs) and photoreceptors, which can be well grown on the laminin modified substrate, which serves as a mimicking subretinal bruchs' basement that can facilitate the growth, phagocytosis, and Pigment epithelium-derived factor (PEDF) secretion of RPE cells. Therefore, the multilayered retinal cell implant of the invention has more potential for successful retinal repair.

In one aspect, the invention provides a method for preparing a multilayered retinal cell implant. The method comprises coating a substrate with laminin to obtain a laminin modified substrate and growing retinal cells derived from stem cells or induced pluripotent stem cells (iPSCs) on the laminin modified substrate, wherein the retinal cells as grown include multiple layers of retinal cells, and provides properties and efficacy facilitating retinal repair.

According to the invention, the retinal cells can be well grown on the laminin modified substrate, and the retinal cells as grown includes multiple layers of various retinal cells, including at least retinal pigment epithelium (RPE) cells and photoreceptors. It is confirmed in the examples that the laminin modified substrate, serving as a mimicking subretinal bruchs' basement that can facilitate the in vitro growth, phagocytosis, and Pigment epithelium-derived factor (PEDF) secretion of RPE cells.

In another aspect, the invention provides the multilayered retinal cell implant as obtained by the method, which is potential for retinal repair.

In a further aspect, the invention provides a method for repairing a retinal defect within an eye of a subject in need thereof, comprising transplanting on the retinal defect the multilayered retinal cell implant obtained by the method.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiment which is presently preferred. It should be understood, however, that the invention is not limited to this embodiment.

In the drawings:

FIG. 1A-1D show the generation of RPE monolayer from patient-specific iPS cells. FIG. 1A provides microscopic photos of AMD patient-specific iPS cells, differentiated RPE cells, and PRE monolayer. Scale bar=100 μm. FIG. 1B provides the image of an immunostaining of RPE specific protein markers, Rpe65, bestrophin, MITF, and PAX, as well as the ZO-1 tight junction marker, in dRPE cells. Scale bar=50 FIG. 1C shows that dRPE cells were subjected to a phagocytosis assay by incubated with pH-sensitive red fluorescence-conjugated E. coli particles for 18 hours. The change of fluorescence dye was observed under fluorescence microscope (left) and quantified by flow cytometry (right). Scale bar=100 μm.

FIG. 1D provides a schematic illustration of the designed procedures for biomimetic subretinal implant.

FIG. 2A-2G show the characterization of plasma modified PDMS shee, FIG. 2A provides a schematic illustration of the surface modification procedure of PDMS. PDMS membranes were generated using medical grade elastomer (Nusil Technology LLC, Inc.), and PDMS surfaces was treated with O₂ plasma. After plasma modification, silane solution (APTES) was used to introduce the amine groups (NH₂) onto the surface for further protein attachment. Laminin was chemically attached on to the aminated surface via EDC/NHS mediated covalent bonding. In the reaction, the carboxylic acid group son laminin enable to couple with the amine group on PDMS surface and finally form a stable amide bond on PDMS substrate. FIG. 2B provides SEM images of PDMS substrate with (10W and 50W) or without plasma treatment for 30 sec. The samples were dried in vacuum and then sputter coated with gold (JFC 1200, JOEL Tokyo, Japan). Images were obtained using a JSM-7600F (JOEL, Tokyo, Japan) SE) with electron beam energy of 5 kV. Small granular-like structure appears on PDMS surface after plasma treated at 10W. Wave structure formed on the surface after plasma treated at 50W. Scale bar=100 nm. FIG. 2C provides SEM images of plasma treated ODMS substrates with different plasma exposure time and power. Scale bar=100 nm. FIG. 2D shows that the chemical composition of PDMS substrates was confirmed using a grazing angle reflectance) (80° FTIR measurement. The characteristic peaks of unmodified PDMS substrate include: symmetric and asymmetric —CH₃ stretching from the ESi-CH₃ group at 2870 and 2970 cm⁻¹, respectively; symmetric —CH₃ bending from the ESi-CH₃ group at 1259 cm⁻¹; —CH₃ rocking from the ESi-CH₃ group at 793 cm⁻¹; Si—O—Si peaks at 1076 and 1018 cm⁻¹. PDMS substrate with plasma treatment are increased the —OH stretching from 3100-3600 cm⁻¹ in the IR spectra. After the reaction of protein attachment completed, the presence of protein on the surface is characterized by the peaks corresponding to the amide I (H—CO—NH₂), amide II (H—CO—NHR), and amide III (H—CO—NHRR′) at 1640, 1550 and 1320 cm-1, respectively. The results confirmed the coating of laminin on plasma modified PDMS surface. FIG. 2E shows that water contact angles on the surfaces of a: unmodified PDMS, b: 02 plasma treated PDMS (PDMS-Pm), c: aminized PDMS-Pm, and d: PDMS-PmL were measured at ambient temperature by a video-image sessile drop tensiometer (top) and quantified in the chart (bottom). Surface roughness and heterogeneous were characterized by dynamic contact angle measurement. The results show the laminin-coated PDMS becomes more hydrophilic and increase surface roughness (reflected by the angle of contact angle hysteresis), resulting in a highly favored surface for cell attachment. FIG. 2F shows that iPSC-differentiated RPE and the ARPE-19 cell lines were seeded on the PDMS, 10W/5-min plasma treated PDMS (PDMS-Pm), and 10W/5-min plasma treated PDMS with laminin coating (PDMS-PmL). The morphology of cells were observed under microscope.

FIG. 2G provides the quantified cytotoxicity (left) and attachment (right) of dRPE and ARPE-19 on PDMS, PDMS-Pm, and PDMS-PmL films.

FIG. 3A-3E show the electron microscopic structure of the dRPE monolayer on PDMS-PmL: FIG. 3A provides the SEM image of porous PDMS (Scale bar=100 μm) (Left panel) and the conducting current of glassine membrane; current response of porous DPMS; current response of plastic membrane; current response of 0.22 μm filter membrane; current response of 3K cut-off dialysis membrane (Right panel). The potentials in this work were measured with direct relevance to the Ag/AgCl reference electrode. FIG. 3B shows the elastic modulus of porous PDMS. FIG. 3C provides a schematic illustration of generating the dRPE/PDMS-PmL biomimetic film. FIG. 3D provides the SEM of the dRPE monolayer on PDMS-PmL film (top left, scale bar=10 μm.) and the typical hexagonal organization of dRPE cells on PDMS-PmL (bottom left, scale bar=10 μm.). SEM image of the apical microvilli of dRPE cells when grown on PDMS-PmL film (top right, scale bar=10 μm.). A TEM image revealed the cellular melanosome deposit of the dRPE cells when grown on PDMS-PmL film (bottom right, scale bar=500 nm.). FIG. 3E shows that dRPE cells on PDMS-PmL film were subjected to a phagocytosis assay by incubated with pH-sensitive red fluorescence-conjugated E. coli particles for 18 hours. The change of fluorescence dye was observed under fluorescence microscope. Scale bar=100 μm.

FIG. 4A-4F show the evaluation the growth and function of dRPE cells on PDMS-PmL: FIG. 4A shows that patient-specific iPSC were seeded on PDMS-control and PDMS-PmL and subjected to the RPE differentiation protocol. The cell morphology at day 15, 20, and 25 were observed under microscope. Scale bar=100 μm. FIG. 4B shows that the differentiated RPE cells on PDMS-control and PDMS-PmL were subjected to an immunostaining for ZO-1. Scale bar=50 μm. FIG. 4C provides Western blot analysis of the RPE specific proteins Otx2 and Mitf in cells undergoing the RPE differentiation protocol at indicated days. FIG. 4D shows that dRPE/PDMS-Control and dRPE/PDMS-PmL were subjected to an ELISA assay to assess the secreted levels of PEDF. FIG. 4E shows the immunofluorescent staining of PEDF on dRPE/PDMS and dRPE/PDMS-PmL biomimetic films. Scale bar=50 μm. FIG. 4F shows that dRPE/PDMS and dRPE/PDMS-PmL were subjected to a phagocytosis assay by incubated with pH-sensitive red fluorescence-conjugated E. coli particles for 18 hours. The change of fluorescence dye was observed under fluorescence microscope. Scale bar=25 μm.

FIG. 5A-5F show the development of PDMS-PmL carried multilayer of iPSC-derived retinal tissues. FIG. 5A provides a schematic illustration of the procedure for producing iPSC/neural progenitor-derived photoreceptor progenitor and dRPE bilayer-coating PDMS-PmL biomimetic film. FIG. 5B provides the immunofluorecent staining for neural progenitor-derived photoreceptor precursor (VSX) and dRPE (RPE65) bilayer co-cultured on PDMS-PmL film. Scale bar=50 μm. FIG. 5C provides SEM analysis of the neural progenitor-derived photoreceptor precursor/dRPE bilayer coated on PDMS-PmL biomimetic film. Scale bar=100 μm. FIG. 5D provides TEM images of the photoreceptor precursor/dRPE bilayer coated PDMS-PmL. Typical morphology of photoreceptors (top left and top right), and the tight junction (bottom left; the arrow tips point at the cell-cell junction) and cellular melanosome deposit (bottom right; pointed by the arrow tips) of dRPE cells were observed. FIG. 5E shows that the GFP-labeled neural progenitor-derived photoreceptor precursor and RFP-labeled dRPE cells on PDMS-PmL film were observed under fluorescent microscope. Scale bar=50 μm. FIG. 5F provides a schematic figure of the iPSC/neural progenitor-derived photoreceptor precursor/dRPE/PDMS-PmL device.

FIG. 6A-6D show that OCT and ocular examination for monitoring long-term biostability of PDMS-PmL in the subretinal space of transplanted porcine. FIG. 6A provides OCT screening demonstrating the well retinal attachment of PDMS-PmL in the subretinal space of transplanted pigs one year after transplantation with retinal surgery. Control: normal retinal without surgery. FIG. 6B and FIG. 6C provides the serial observation of funduscopic photography showed that PDMS-PmL implants at the same anatomical position without inducing any complication for at least 12 months after transplantation. FIG. 6D shows that six weeks after implantation, scotopic ERG responses recorded in PDMS-PmL transplanted eyes were no significantly different from that recorded in control eyes.

FIG. 7A-7F show the long-term biosafety and biostability of the PDMS implant and PDMS-PmL implant in vivo, wherein the implantation of PDMS and PDMS-PmL in the subretinal space around macular area in 4 and 6 porcine eyes, respectively. FIG. 7A shows OCT imaging in PDMS-PmL eyes, demonstrating that retinal anatomy was well integrated and the films were placed successfully and maintained stably in the subretinal space of pigs after two-year transplantation. FIG. 7B shows that the retina and RPE on both sides of the implant look unaffected. Around the PDMS-PmL subretinal implantation area, the retinal vasculature was preserved without signs of fibrosis or atrophy by color fundi photography at 2 year. FIG. 7C shows that after 2-year transplantation, the results of scotopic-ERG recordings revealed the retinal function to light response in PDMS-PmL transplanted eyes were no significantly different from that recorded in the before surgery eyes or control eyes. Collectively, these results confirmed the long-term biostability and biosafety of the PDMS-PmL in-vivo. FIG. 7D, FIG. 7E and FIG. 7F show the comparison on the secretion of PEDF by PDMS implant and PDMS-PmL implant between the treatment group and control. It was unexpectedly observed that the PEDF secretion of PDMS implant was inhibited but that of PDMS-PmL implant was not inhibited.

FIG. 8A-8B show that multifocal ERG recordings for monitoring long-term function of PDMS and PDMS-PmL in the subretinal space of transplanted porcine. At each time points, the right panels are 3D-topographical maps, and the right panels are trace array for the individual recordings of mtERG. FIG. 8A shows that after 2-year PDMS transplantation in right eye, mfERG recordings were marked depressed comparing to baseline right eye before surgery. The left eye also serves as a control. FIG. 8B shows that after 2-year PDMS-PmL transplantation in right eye, mfERG recordings showed no significant change comparing to baseline right eye before surgery. The left eye also serves as a control.

FIG. 9 provides a schematic flow-chart of the potential application of dRPE/PDMS-PmL device in AMD patients.

### DESCRIPTION OF THE INVENTION

It is discovered that a substrate modified with laminin provides good properties for growing retinal cells derived from stem cells or induced pluripotent stem cells (iPSCs) thereon. The substrate modified with laminin serves as a mimicking subretinal bruchs' basement, on which the retinal cells were grown, and the retinal cells as grown include multiple layers of retinal cells, and provides good properties and efficacies facilitating retinal repair.

In the invention, the substrate is a sheet of any biocompatible polymeric compound, including but not limited to polydimethylsiloxane (PDMS), poly(methyl methacrylate) (PMMA), Glycidoxypropyltrimethoxysilane (GPTES), aminopropyltriethoxysilane (APTES), Xylene or acetone. One example of the substrate used in the invention is polydimethylsiloxane (PDMS).

According to the invention, the substrate may be modified by laminin in any manner known and/or commonly used in the prior art. In one example of the invention, the substrate is subject to a surface modification with laminin by chemical and oxygen plasma treatment.

It was unexpectedly found in the invention that the substrate, such as PDMS, modified with laminin provides a subretinal environment for dRPE-monolayer grown on the substrate.

In one example of the invention, the PDMS treated with a plasma -PmL (“PDMS-PmL”) provide the following unexpected properties or efficacies:
(1) enhancement of the attachment, proliferation, polarization, and maturation of dRPEs;
(2) increase of the polarized tight junction, PEDF secretion, melanosome pigment deposit, and phagocytotic-ability of dRPEs;
(3) the capability to form multilayer structure with dRPEs and photoreceptor-precursors;
(4) well-biocompatibility;
(5) maintenance of trophic PEDF secretion.

Taken together, the laminin modified substrate, such as PDMS-PmL, is able to sustain the physiological morphology and functions of polarized RPE monolayer, and expresses the potential of rescuing macular degeneration in vivo.

The present invention will now be described more specifically with reference to the following examples, which are provided for the purpose of demonstration rather than limitation.

### EXAMPLEs

### Example 1

### Preparation of PDMS-PmL Sheet and Evaluation

Phagocytosis Assay

Phagocytosis is assessed by a flow cytometry-based method using pHrodo™ E. coli fluorescent bioparticles (Invitrogen) which fluoresce when internalized in the reduced pH environment of intracellular phagosomes. Bioparticles do not fluoresce at neutral pH, therefore background fluorescence related to nonspecific adherence is negligible. Bioparticles were prepared as the concentration of 5 μg/_82 L in Live Cell Imaging Solution (Invitrogen) according to the manufacturer's instructions. Confluent RPE were incubated with 70μL bioparticles plus 630 μL HBSS per one well of a 12-well plate in CO2 -independent medium (Invitrogen) for 17-18 hours at 37° C. Negative control plates were incubated at 4° C. . Cells were examined under the microscope, harvested by TrpLE and analyzed by flow cytometry counting 20,000 events on a Flow Cytometer. Positive uptake by phagocytosis is indicated by a rightward shift in fluorescence intensity on histogram plots of the gated cell population.

PDMS Surface Modification

The process of PDMS surface modification is consisted with three steps as follows:
1) PDMS oxidation via plasma treatment (PDMS-OH). The samples were exposed to oxygen plasma (PC150, JunSun Tech Co., Ltd) to create a hydrophilic surface. They were treated with oxygen plasma at 10⁻² Torr for 5 mins at 50W, and an oxygen flow rate of 17 sccm.
2) Aminization of PDMS substrates (PDMS-NH₂). After plasma treatment, the PDMS membranes were immersed in a silane solutions of 1% by volume APTES (Ca.440140, Sigma-Aldrich, Mo) in absolute ethanol. Then, 5% of by volume DI water was added to the solution to hydrolyze the silane and allowed to react for 15 min at 75° C. The PDMS samples were washed once with 75% by volume aqueous ethanol and the three times with DI water following the silane reaction to remove residual silane compounds. The aminized PDMS membrane is denoted as PDMS-NH₂.
3) Surface grafting of laminin onto PDMS-NH₂ membrane. Conjugation of laminin on PDMS-NH₂ membrane was performed by crosslinker EDC/NHS (Sigma-Aldrich, Mo). EDC/NHS (1:1 molar ratio) were added to 10 μg/ml laminin in PBS buffer to obtain a final concentration of 10mM, and allowed to react with PDMS-NH₂ membrane for 1 hr at 37° C. The PDMS membranes were then washed by DI water to remove residual reagents, and rinsed by PBS before the cell seeding.

Contact Angle Measurement

Water contact angle on PDMS surfaces were measured at ambient temperature by a video-image sessile drop tensiometer (Model 100SB, Sindatek Instruments Co., Ltd). A 1.5 μl drop of DI water was dropped on the substrate surface and photographed. The shape of the drop and baseline was then fitting by conic section analysis to calculate the three phase (solid-liquid-gas) contact point. For each PDMS substrate, the measurements were performed on five different areas of the surface and the values were averaged.

Determination of Amine Content on Surface by Colorimetric Assays

The amount of exposed amine on the silanized PDMS surface (PDMS-NH₂) was quantified using a colorimetric method—Acid Orange II assay. In brief, aminized PDMS samples (3.9 cm² in 12 well culture dish) were immersed in 1 mL of acid orange dye solution (500 μM) in acid condition (Milli-Q water adjusted to pH 3 by 6N HCl) overnight at room temperature. The samples were then washed 3 times using the acidic solution (pH3) to remove unbound dye. After that, the colored samples were immersed in lmL of alkaline solution (Milli-Q water adjusted to pH 12 by 6N NaOH) overnight to allow the bound dye on substrates to detach. The amount of the bound dye, representing the amount of surface accessible amine, was quantified by measuring the optical density at 492nm. Different concentration of Acid Orange II solution (10-50 μM) were prepared in Milli-Q water and adjusted to pH 12 to establish the standard curve. Unmodified PDMS substrate served as negative control.

Implantation of dRPE/PDMS-PmL in the Subretinal Space of RCS Rats

All experimental animals were raised in the Animal Center of Taipei Veterans General Hospital and all surgical procedures were performed in accordance with the institutional animal welfare guidelines of National Laboratory Animal Center. The dRPE/PDMS-PmL implants were transplanted into 20-˜24-week-old Royal College Surgeon (RCS) rats' eyes for short-term and long-term observation. After topically applying 0.5% tropicamide eye drops in the treated eyes, these rats were anesthetized with 0.05 ml/100 g Rompun (Bayer, Taiwan) and 0.1 ml/100 g Zoletil (Virbac, Taiwan) through intraperitoneal injection. Peritomy of superior conjunctiva were done and the rats' eyeballs were temporally fixed using the 6-0 silk sutures in the beginning of surgery. The sclera and choroid of treated area was then incised using a 26 gauge needle. After dissecting the subretinal space by injecting some viscous fluid, the implant was inserted into the exactly subretinal space through this sclera-choroid wound. At the end stage of the surgery, conjunctival wounds were closed with 10-0 sutures and the grafted eye was applied and covered with 0.3% gentamicin eye ointment. Visualized color fundus, OCT images, and ERG for functional determination in all studies rats were followed and recorded.

Statistical Analyses

Results are expressed as mean±SD. Differences between the groups were analyzed using one-way ANOVA followed by Student's t test. A P-value <0.05 was considered statistically significant.

Results

1 Generation of Pluripotent Stem Cell-Derived RPE Monolayer

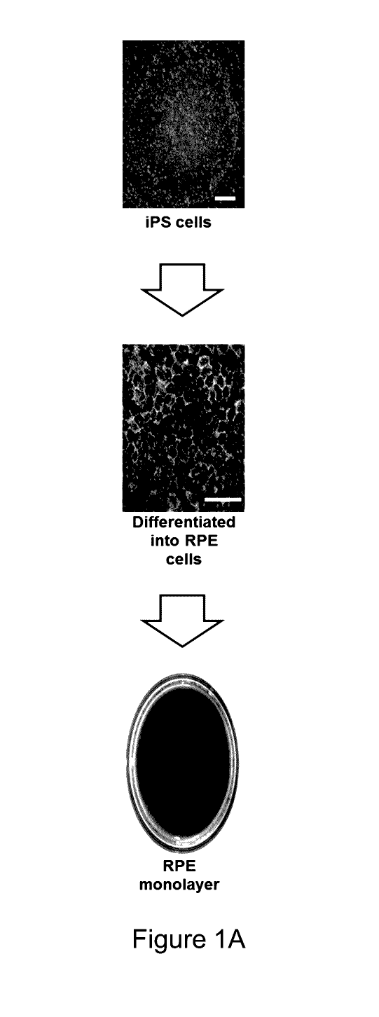

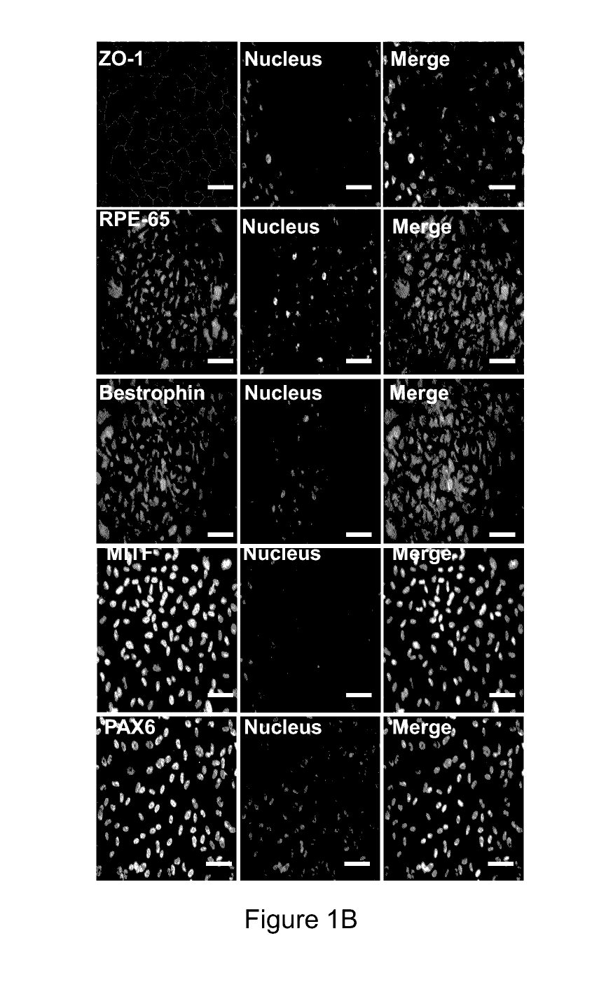

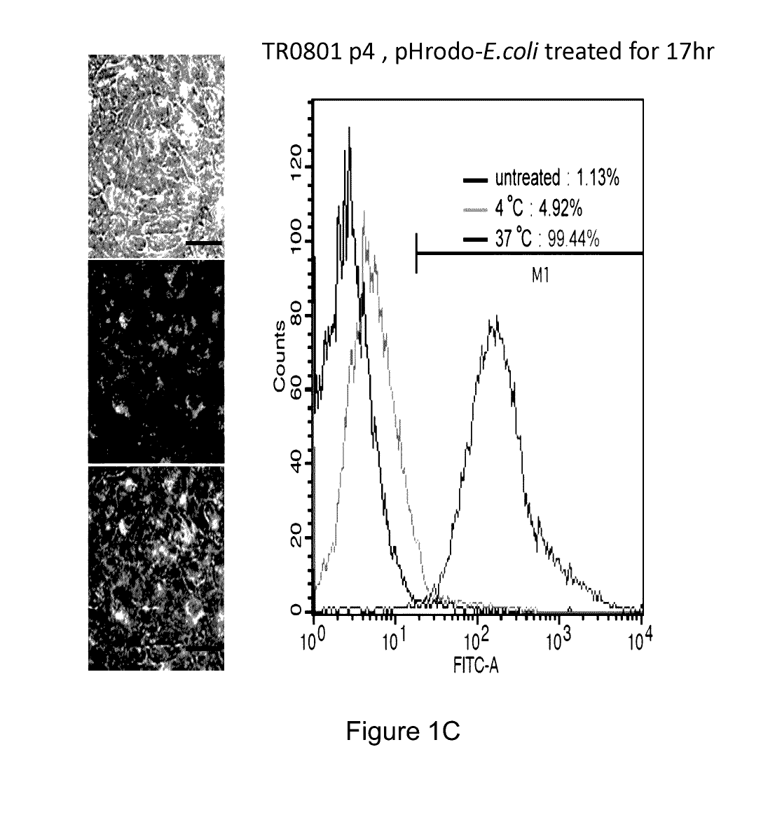

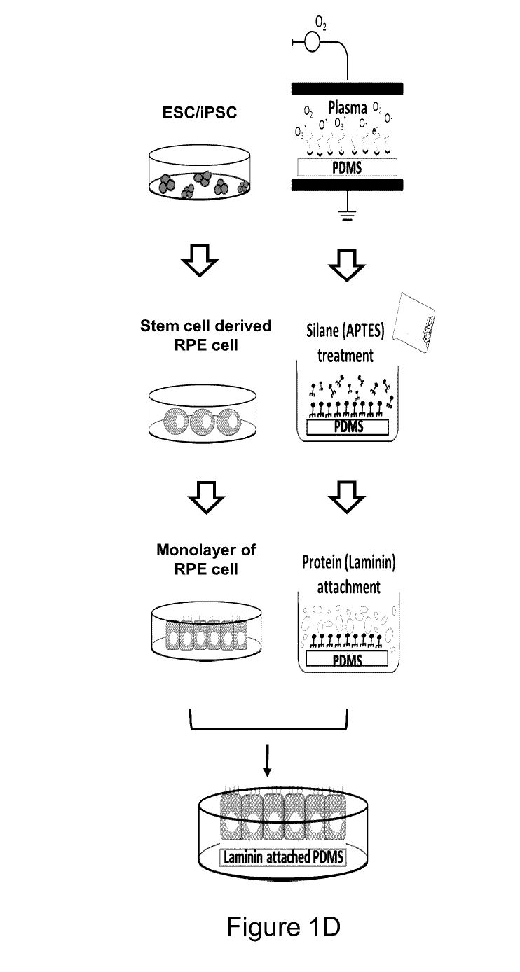

Pluripotent cell-derived RPE has been used in the repair of retina disease in several animal models, as well as tested in pre-clinical trials for repairing the degenerated RPE in advanced AMD patients. We previously established human iPSC cell lines from T-cells through delivering Oct4, Sox2, K1f4, Lin28, Myc, and sh-p53 by electroporation (FIG. 1A, top, Suppl. Information). The human iPSCs were then differentiated into RPE cells (FIG. 1A, middle, Suppl. Information) for further in vitro and in vivo studies. These pluripotent cell-differentiated RPE (dRPE) presented hexagonally-packed morphology with heavy pigmentation (FIG. 1A, middle and bottom), and expressed RPE specific protein markers such as RPE65, bestrophin, MITF, and PAX6, as well as the zodula occludens-1 (ZO-1), a tight junction-specific protein (FIG. 1B). To examine the phagocytosis function of the dRPE cells, we incubated dRPE cells with the pH-sensitive pHrodo™ E. coli fluorescent bioparticles to visualize the engulfment of phagosomes. As shown in FIG. 1C, dRPE cells expressed high level of red fluorescence, which is induced when cells undergo phagocytosis and engulf the particles in phagosomes. Quantification of the red fluorescence revealed significant enhanced phagocytotic activity in dRPE cells, compared with control (FIG. 1C, right). Collectively, these analyses confirmed that dRPE cells possessed typical RPE morphology, markers expression, phagocytosis function, and tight junction of cell contact. Furthermore, physiological morphology of human RPE is a polarized monolayer lining with under the layer of photoreceptors to provide essential nutrients and engulfs the tips of photoreceptor outer segments. It is critical for RPE cells to maintain their physiological organization to improve their survival and exert their functions. We then designed a PDMS-based biomimetic film aiming to support the polarized dRPE monolayer for implantation in subjects' subretinal space in vivo (FIG. 1D).

2 PDMS Modification with O₂ Plasma Treatment and Laminin Coating.

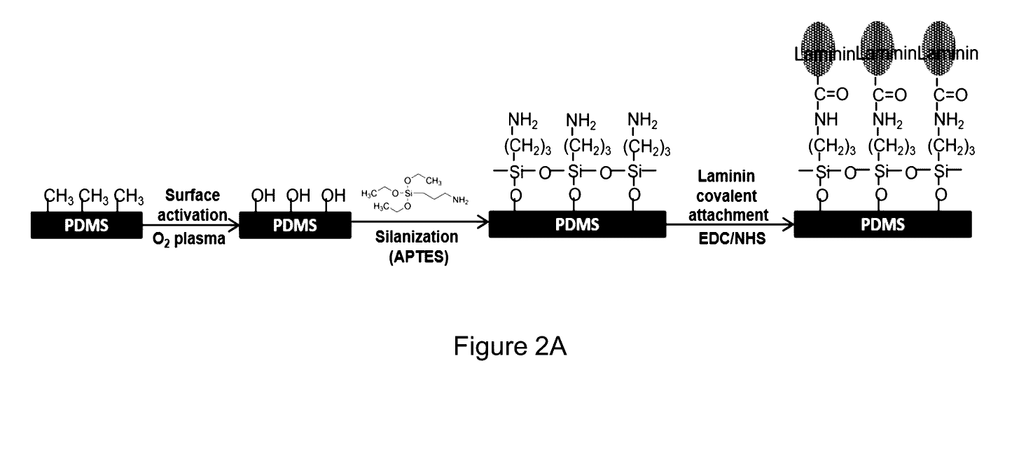

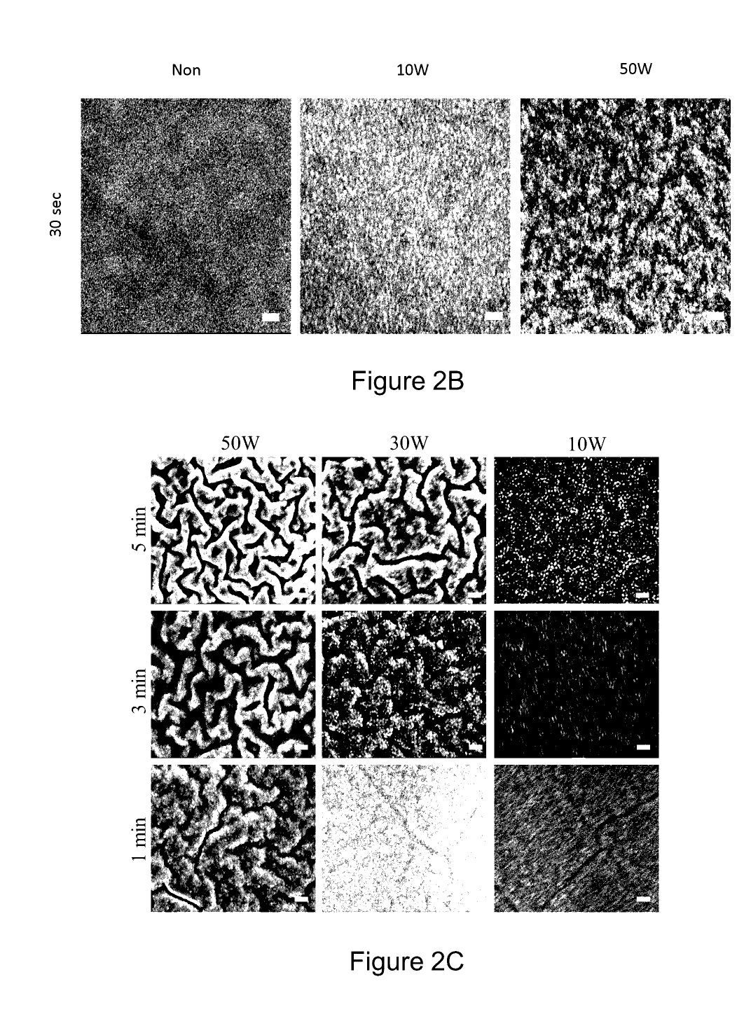

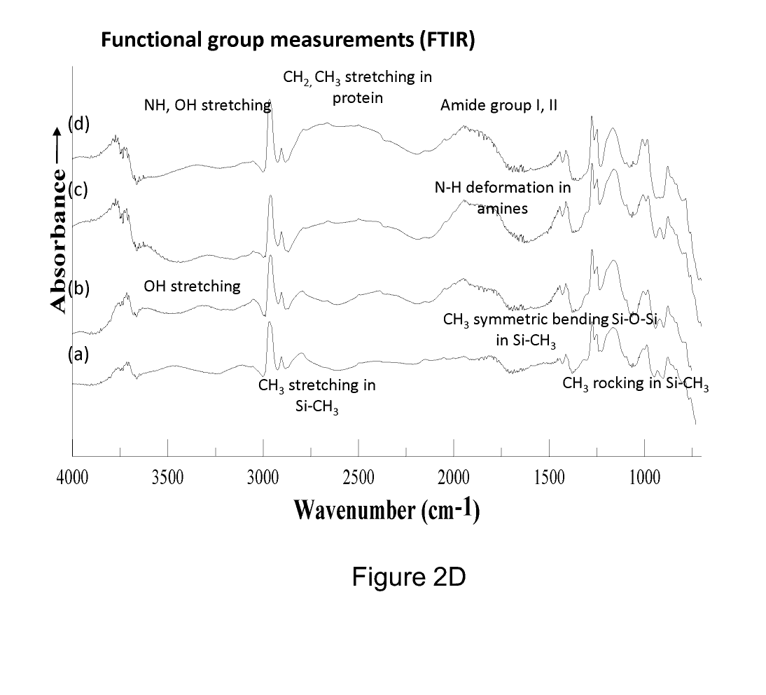

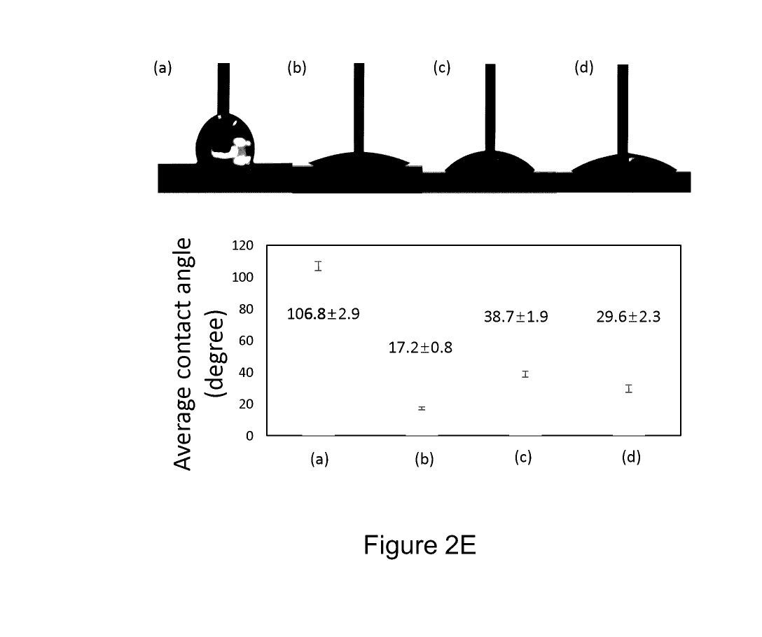

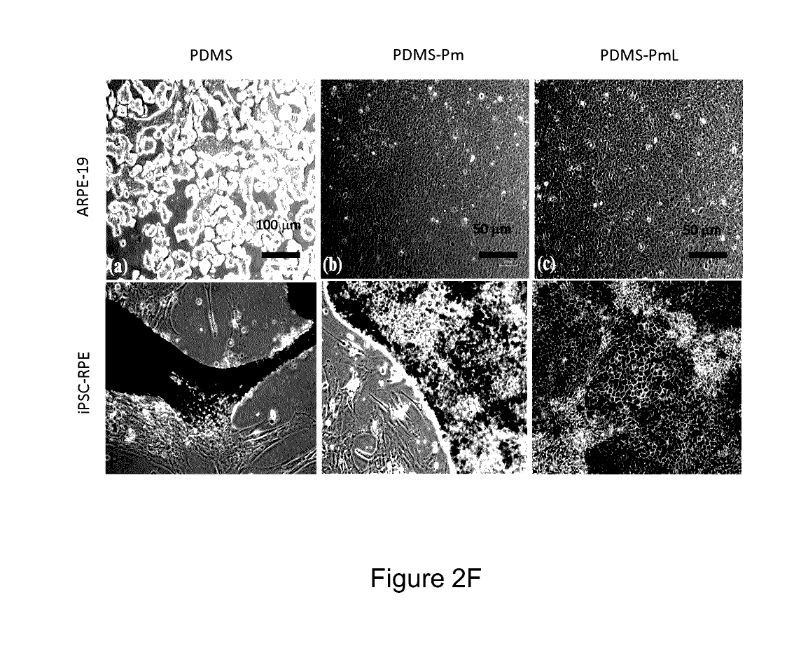

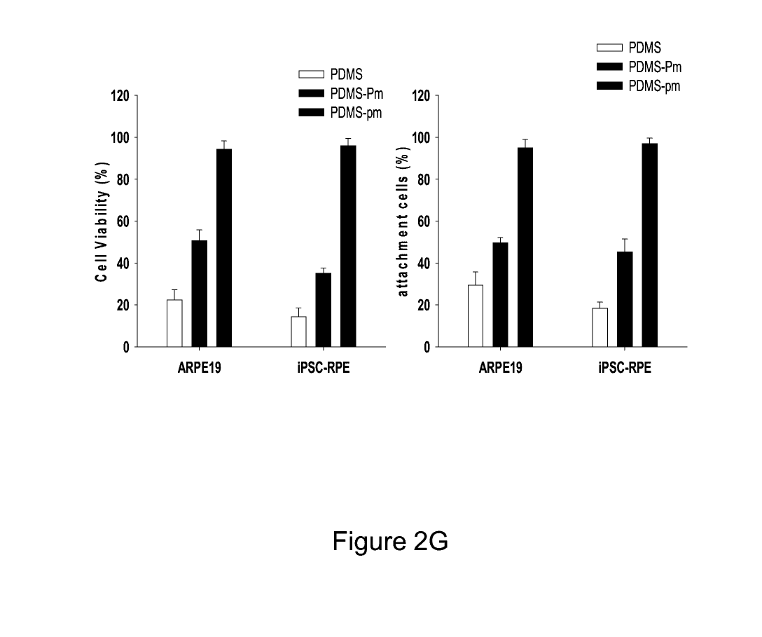

The PDMS is a popular bio-safe material with high bio-compatibility that has been widely used for microfluidic device construction, especially for biological application. However, its low cell adhesiveness and high hydrophobicity causes its major drawbacks and results in substantial sample loss. In order to overcome the disadvantages of PDMS, we executed surface modification on PDMS substrates to enhance its adhesiveness and reduce its hydrophobicity. As shown in FIG. 2A, the surface modification scheme used to functionalize PDMS substrate. We first pre-treat PDMS surface with O₂ plasma to introduce the OH group, as described in previous studies. PDMS surfaces then became hydrophilic after the O₂ plasma treatment due to the presence of silanol (Si—OH) group on the surface. The morphology of PDMS membrane before and after 30 sec of O₂ plasma treatment was observed by scanning electron microscopy (SEM) (FIG. 2B). The plasma-treated PDMS expressed granular surface in comparison to the homogenous non-treated PDMS. The oxygen content on the PDMS surface would increase along with both the increased plasma power and prolonged exposure time; however, this may also cause etching on the surface and increase the surface roughness on PDMS substrate, which affects the subsequent cellular attachment on the surface. To evaluate the optimal condition of PDMS modification, PDMS films were treated by O₂ plasma with serial titrations of power and different exposure times (FIG. 2C). The PDMS surface were significantly etched when treated with 50W and 30W of O₂ plasma, whatever the exposure time was (FIG. 2C). When the plasma power was reduced down to 10W, the PDMS surface appeared to maintain its integrity (FIG. 2C). With a serial test of low-power plasma treatment, we found the 10W/5-min treated PDMS seems to be maintaining better quantity for treating silanization and laminin-coating on PDMS as compared to others (data not shown). Hence, the 10W/5-min plasma modification condition was chosen as the standard treatment for this study. Using a grazing angle reflectance) (80° FTIR, the surface characteristics of unmodified, plasma treated, surface aminized, and laminin-coating/plasma-treated PDMS (PDMS-PmL) were shown in FIG. 2D, and the presence of laminin on PDMS-PmL surface was proved. To assess the hydrophilic property of the modified PDMS, we measured the water contact angle on the surfaces of PDMS with different modification, and demonstrated that the PDMS-PmL presented increased hydrophilic property compared with non-modified PDMS control (FIG. 2E). Moreover, we examined the effect of laminin-coating on cell adherence to PDMS. The results showed that both dRPE cells and ARPE-19 RPE cell line formed a hexagonal monolayer on PDMS-PmL but less homogenous on PDMS-Pm (O₂ plasma only without laminin-coating), while both cells had difficulty to attach on the non-modified PDMS (FIG. 2F). We then examined the cytotoxicity and attachment of dRPE and ARPE-19 on PDMS, PDMS-Pm, and PDMS-PmL. As shown in FIG. 2G, both cells had better survival and highest percentage of attachment when grown on PDMS-PmL, compared with non-modified PDMS and PDMS-Pm. Taken together, these analyses showed that the modification with plasma treatment and laminin coating increased the hydrophilic property of PDMS, allowing a better growth and attachment of dRPE cells.

3 Ultrastructure of the dRPE Monolayer on PDMS-PmL.

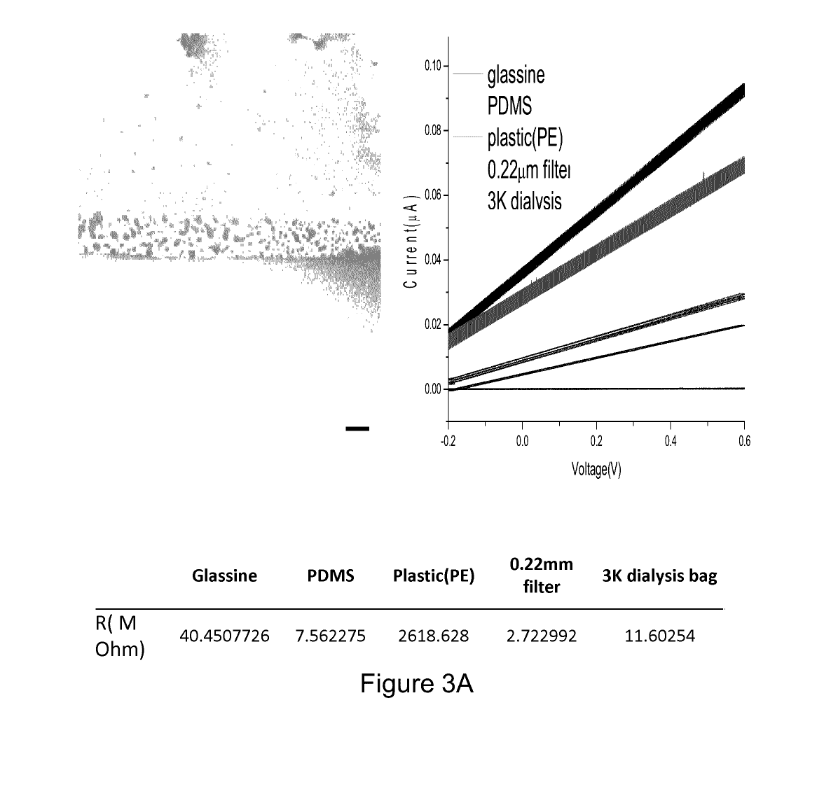

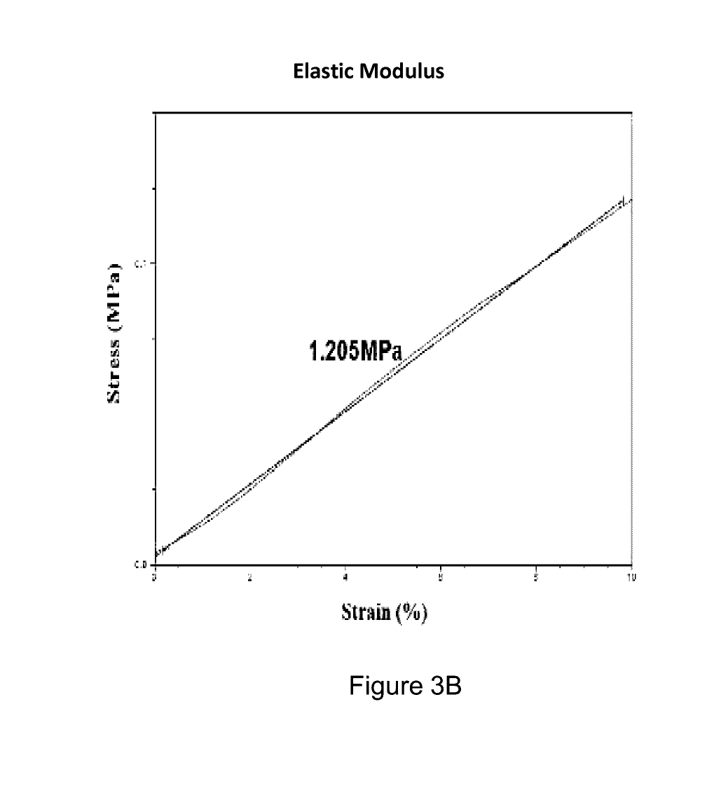

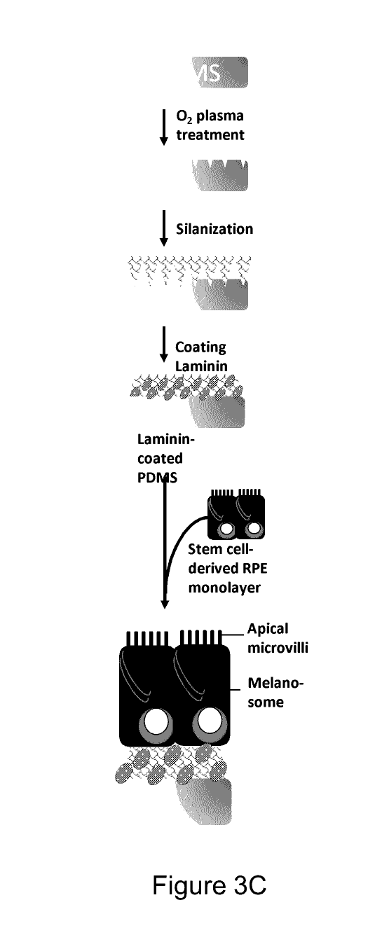

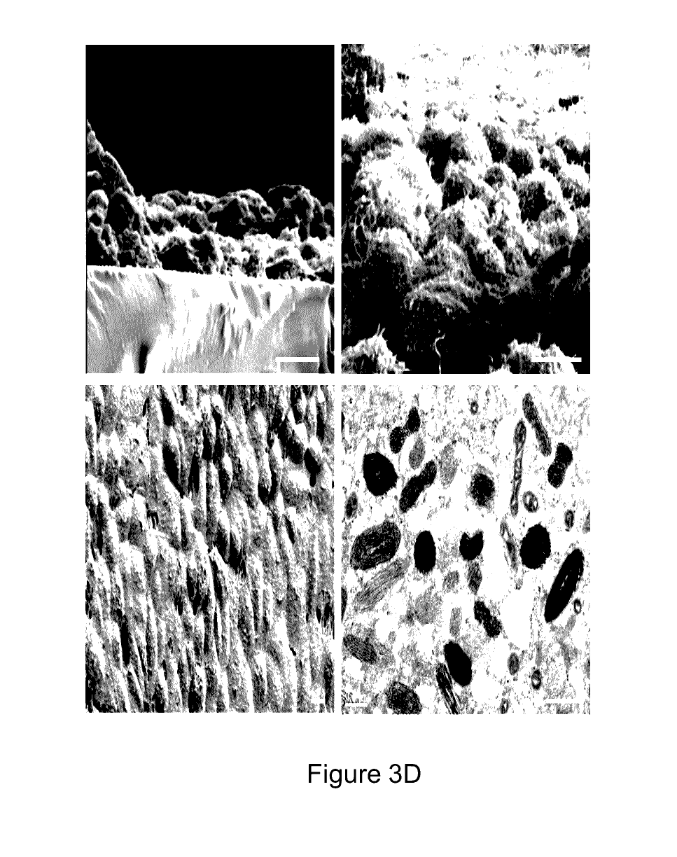

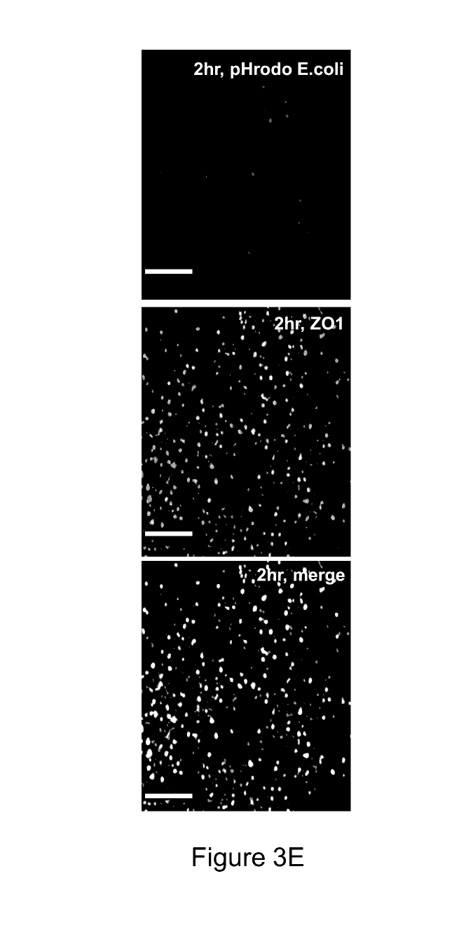

We then evaluated if this modification affect the conductance and diffusing ability of PDMS. As shown in the left panel of FIG. 3A, PDMS-PmL still presents the multi-porous characteristics. By using modified electrochemical spectroscopy analysis (Suppl. Information), the conductance and diffusing ability of this porous PDMS-PmL is better than glassine and PE membrane, but the effective pore size may be smaller than 0.22 μm filter (Merk Millipore corp., Darmstadt, Germany) and 3.5K cut off dialysis membrane (Cellu.Sep, Membrane Filtration Products, InC., Seguin, Tex., USA) (FIG. 3A, right). These results indicate that pore size of porous PDMS-PmL is larger than sucrose molecule (˜9 Å) which can penetrate the glassine membrane but smaller than 30 amino acid peptides (˜2 nm). Therefore, the effective pore size of our porous

PDMS-PmL is approximately from 0.9˜2 nm. Moreover, the result of elastic modulous examination also indicated that the modification did not affect the elasticity of PDMS (FIG. 3B). Taken together, these data suggested that the small molecules, including essential-nutrients like glucose, sucrose, amino acids, and small peptides, could directly pass through PDMS-PmL to provide an implanted environment for maintaining the survival of dRPE cells.

To further examine whether PDMS-PmL facilitates the polarization of dRPE, we seeded the dRPE cells on PDMS-PmL as illustrated in FIG. 3C. Using scanning electron microscope (SEM), the ultrastructural morphology of dRPE surfaces and the coating of dRPE monolayer on PDMS-PmL were visualized. The surface of PDMS-PmL appears smooth and compact with significant integrity, avoiding cells to migrate through the membrane (FIG. 3D).

In SEM images, the formation of a flat and polarized RPE monolayer was observed on PDMS-PmL (FIG. 3D, top left). These dRPE cells also appeared to retain a uniform hexagonal shape (FIG. 3D, bottom left) and abundant apical microvilli on the laminin grafted PDMS-PmL (FIG. 3D, top right). Using transmission electron microscope (TEM), we showed that these dRPE cells expressed melanosome pigment deposit, a typical character of mature RPE cells (FIG. 3D, bottom right). Moreover, pHrodoTM E. coli phagocytosis assay (FIG. 3E, top) and immunofluorescent staining of ZO-1 (FIG. 3E, middle) demonstrated active phagocytosis function and tight junction of cell-cell contact in the dRPE monolayer (FIG. 3E, bottom). Taken together, our findings indicated that the surface of modified PDMS-PmL membrane provides a basement membrane-like environment for RPE growth and polarization with tight-layout in monolayer structure, the physiological morphology of RPE layer.

4 PDMS-PmL Enhanced the Differentiation and Functional Maturation of dRPE Cells.

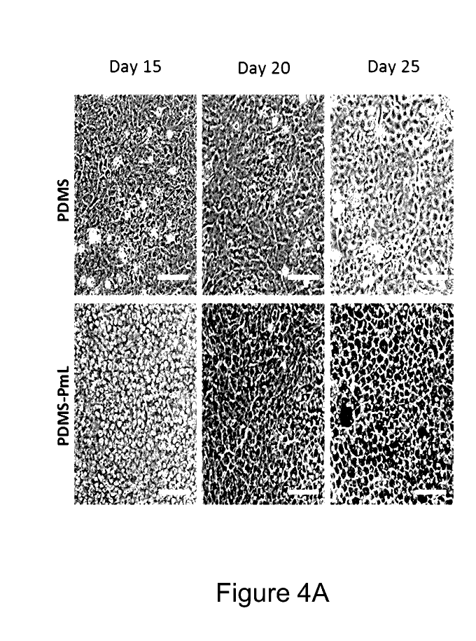

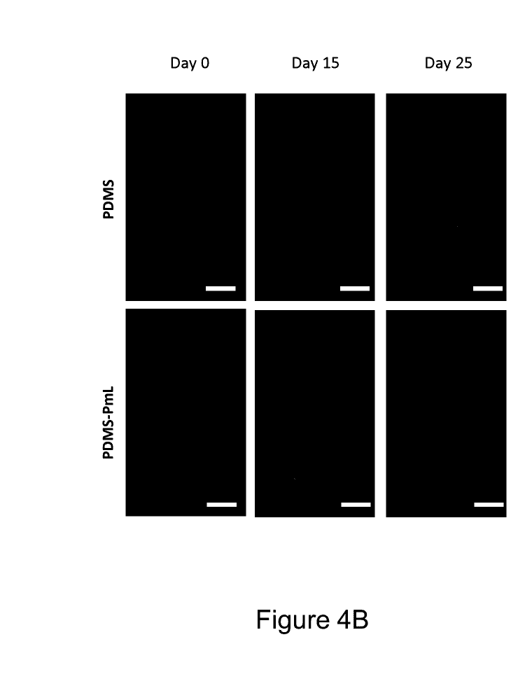

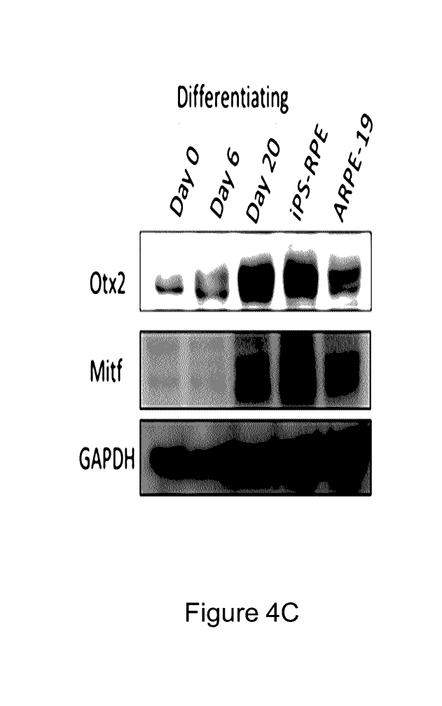

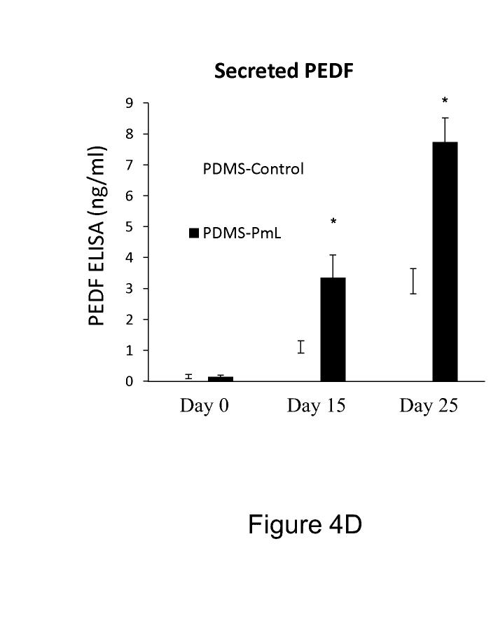

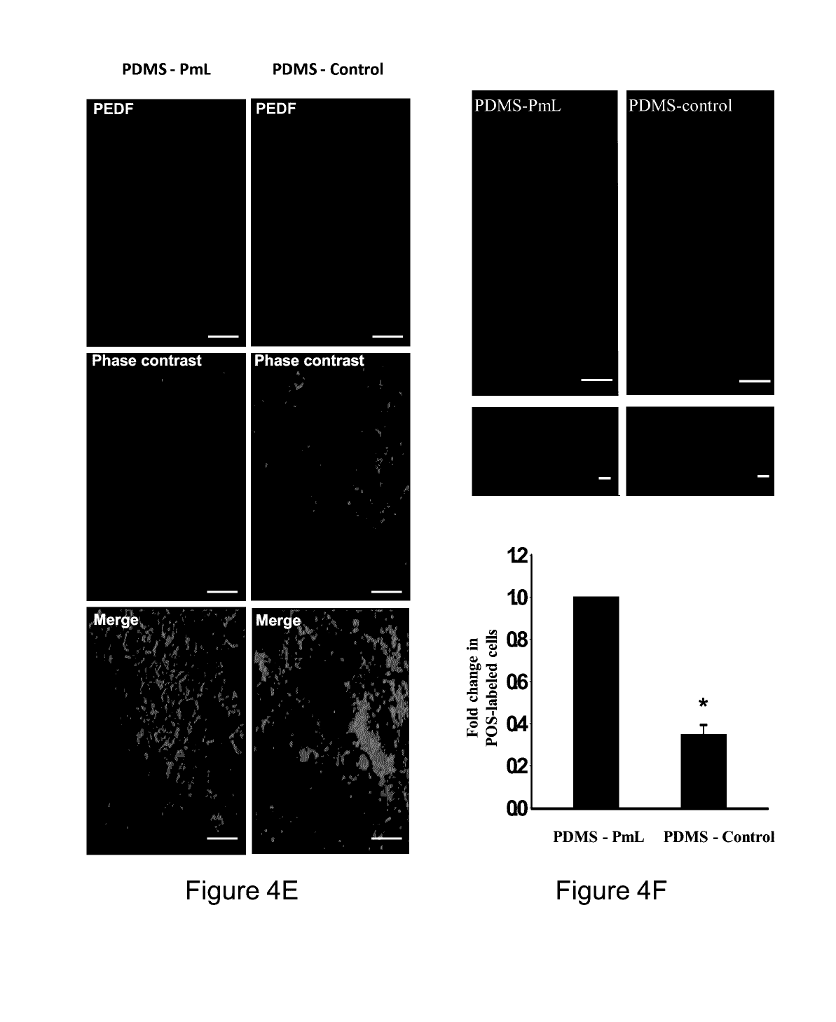

Laminin is one of the important components of the retinal extracellular matrix, as well as the microenvironment niche for stem cell differentiation. To further explore whether PDMS-PmL facilitates RPE differentiation and maturation, human iPSCs were seeded on PDMS and PDMS-PmL before undergoing RPE differentiation protocol. Observation of the dRPE cells under microscope showed that during the 25 dyas of differentiation, cells on PDMS-PmL presented better attachment and hexagonal organization compared with cells on PDMS (FIG. 4A). Moreover, dRPE cells on PDMS-PmL expressed more melanosome pigment deposit since day 20 of the differentiation procedure (FIG. 4A). Importantly, immunofluorescent staining of the tight junction-specific ZO-1 protein demonstrated a better organization of ZO-1 at the cell-cell contact of dRPE cells seeded on PDMS-PmL than that on PDMS-control from day 15 after the induction of differentiation (FIG. 4B). Western blot analysis of the iPSCs under on-film RPE differentiation protocol demonstrated a gradual increased expression of the Otx2 retinal-lineage marker and the Mitf RPE-specific protein (FIG. 4C). Notably, the RPE differentiation procedure, which normally takes 30 to 40 days under standard protocol, spent only 25 days on PDMS-PmL to generate mature RPE cells (FIG. 4A and FIG. 4B). These data suggested that PDMS-PmL may provide a niche for efficient RPE differentiation. Moreover, we further evaluate the functional maturation of the dRPE cells on PDMS-PmL by analyzing PEDF secretion level and phagocytosis capability. The ELISA analysis demonstrated an increase of secreted PEDF in the culture medium of dRPE/PDMS-PmL, compared with dRPE/PDMS-control at day 15 and day 25 post-differentiatively. (FIG. 4D). Immunofluorescent staining of extracellular PEDF also supported that PDMS-PmL enhanced the PEDF secretion of dRPE cells (FIG. 4E). In line with increased PEDF secretion, dRPE presented a better phagocytosis activity on PDMS-PmL than growing on PDMS-control (FIG. 4F). All together, our molecular, morphological, and functional analyses confirmed that the modification PDMS with plasma treatment and laminin coating facilitated the differentiation and functional maturation of RPE cells, at least in stem cell-based retinal-lineage differentiation.

5 PDMS-PmL is Able to Carry Multilayers of Retinal Cells.

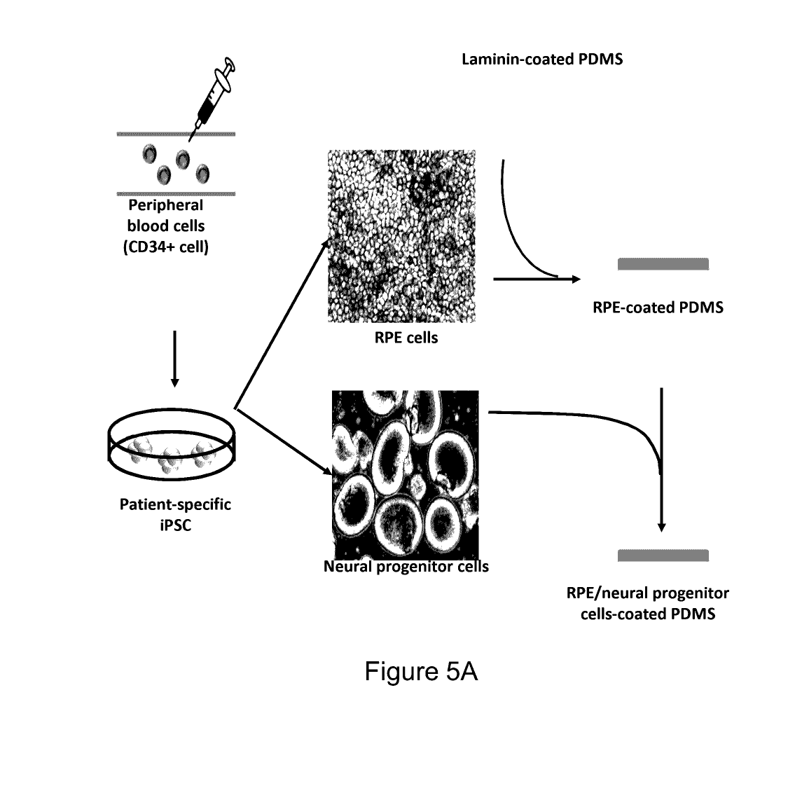

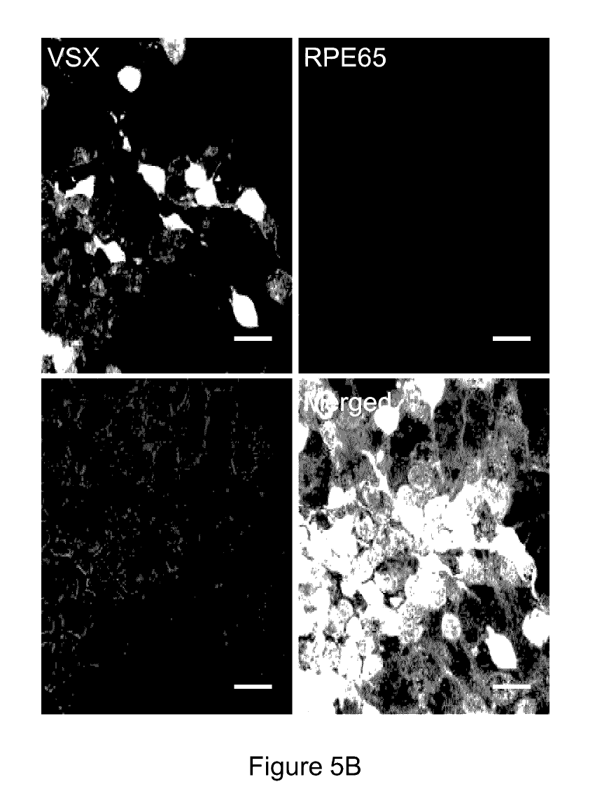

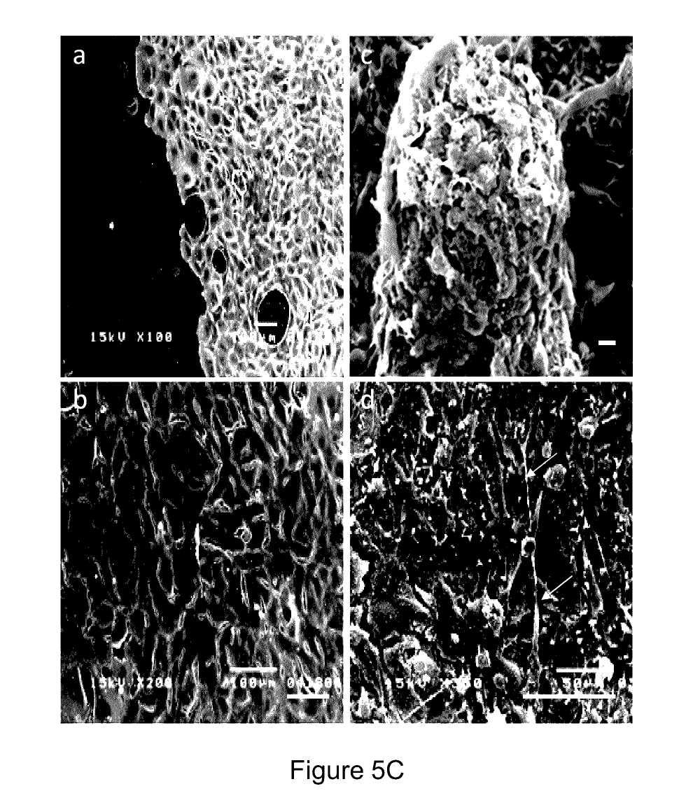

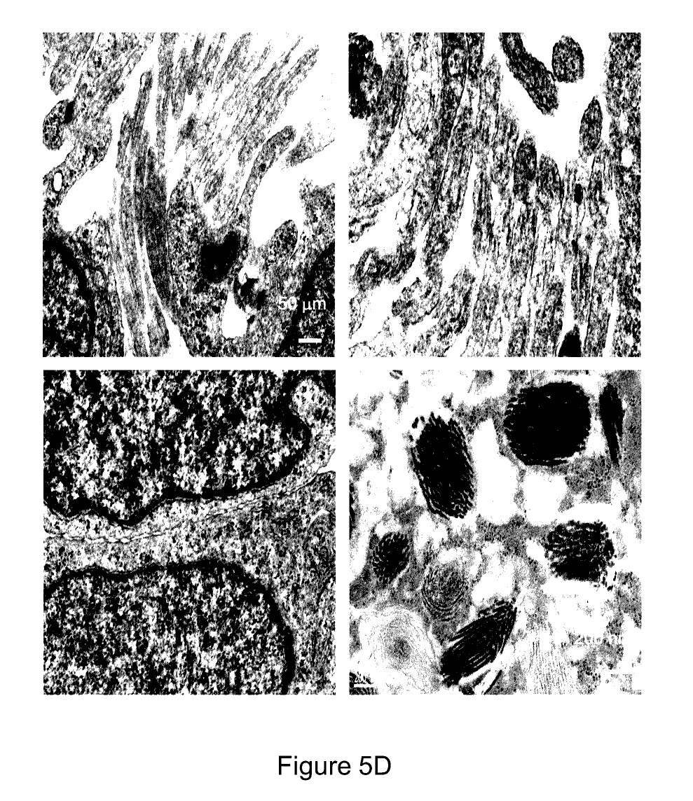

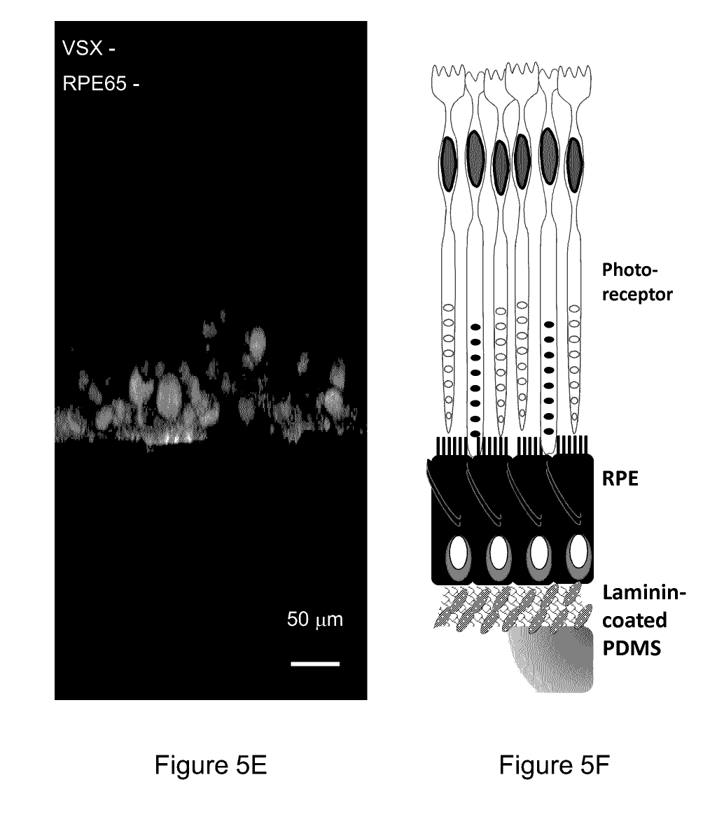

Retina is a complex combination of tissues consisted of several different layer of cells including RPE, photoreceptor, bipolar retinal nerve cells, and retinal ganglion cells. The generation of photoreceptor cells for use in conjunction with the RPE graft would be a solution for recovering the visual dysfunction in severe retinal degeneration. In an attempt to mimic physical structure of retina and examine the diverse applications of PDMS-PmL in severe retinal degeneration like late-stage AMD, we examined the capability of PDMS-PmL membrane to carry multilayer of retinal cells (FIG. 5A). Following our previous protocol, we used dRPE-monolayer as a retinal cell-to-cell environment to facilitate stem cell differentiation toward neural progenitor cells [35]. We then developed a photoreceptor/dRPE/PDMS-PmL multilayer device by seeding the iPSC-derived neural progenitor cells on the hRPE-coated PDMS-PmL membrane. (FIG. 5A). Immunofluorescent staining of the bilayer device showed VSX-positive photoreceptor progenitor cells and RPE65-positive RPE cells co-cultured on the PDMS-PmL film (FIG. 5B), indicated a successful coating of iPSC/neural progenitor-derived photoreceptor precursor and dRPE cells in our system.

Retina function is largely relied on the order and organization of each layer of tissue. We further investigate the ultrastructure of the iPSC/neural progenitor-derived photoreceptor precursor/dRPE bilayer on the PDMS-PmL film. SEM data revealed a monolayer of dRPE on the PDMS-PmL film (FIG. 5C, a-b); on top of the dRPE monolayer laid the differentiated neural progenitor cells with typical spiky morphology (FIG. 5C, c-d). Moreover, electron microscopy demonstrated the typical photoreceptor morphology of the iPSC/neural progenitor-derived photoreceptor precursor cells on the bilayer device (FIG. 5D, top left and top right). The melanosome deposit in the dRPE cells on the bilayer device was also observed (FIG. 5D, bottom right). Notably, tight junction between cell-cell contact can be observed under TEM, suggesting the integrity of the bilayer tissue on PDMS-PmL film (FIG. 5D, bottom left). Furthermore, fluorescent microscope clearly demonstrated a bilayer of RFP-labeled hRPE and GFP-labeled photoreceptor precursor cells on the PDMS-PmL biomimetic film (FIG. 5E, FIG. 5F). These data suggested the capability of PDMS-PmL to carry multilayer of retinal cells and its potential applications in advanced retinal degeneration diseases.

6. Validation of Long-Term Biosafety and Biostability of PDMS-PmL Implant in the Subretinal of Pigs.

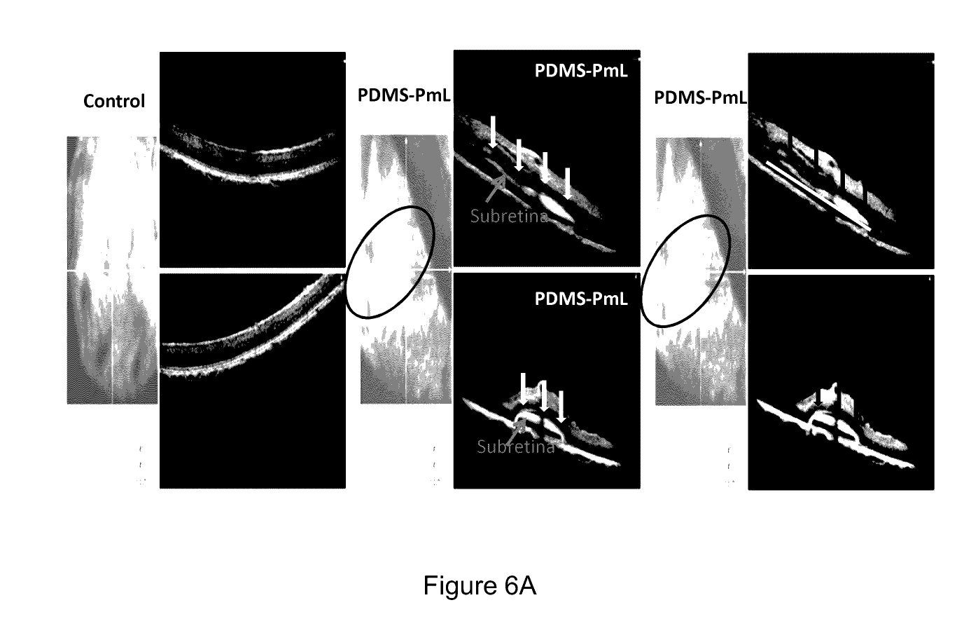

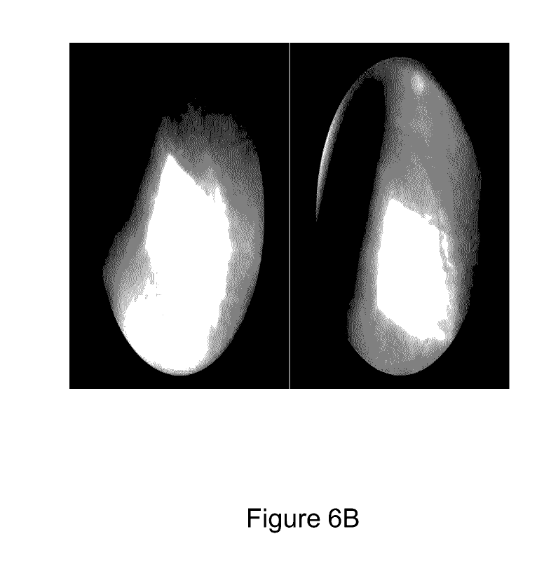

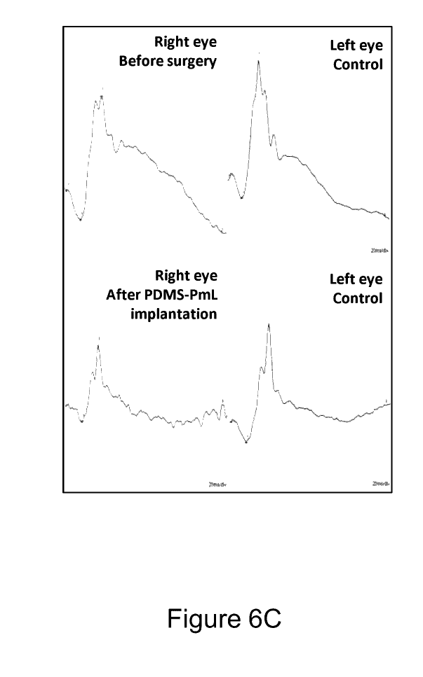

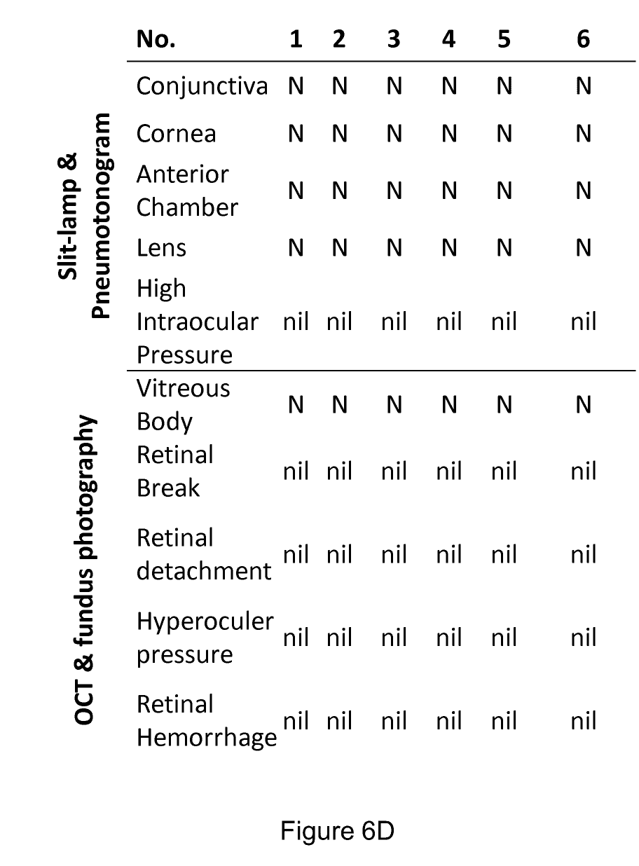

To further validate the long-term biosafety and biostablility of the PDMS-PmL implant in vivo, we performed the implantation of PDMS and PDMS-PmL in the subretinal space around macular area in 4 and 6 porcine eyes, respectively. After the subretinal-transplantation of PDMS and PDMS-PmL, the retinal anatomical structure, function and ocular condition of each subject were routinely checked up every 3 months by optical coherence tomography (OCT), slit-lamp examination, color fundi photography, full-field and multifocal electroretinograms (ERGs). During the 2-year follow-up, the position of the implant and the preservation of retinal structure were monitored by OCT and color fundi photography. In all PDMS and PDMS-PmL-transplanted eyes, the implant was in situ and stable without movement. In PDMS eyes, the cross-sectional OCT imaging identified photoreceptor/RPE layer disrupted and loss subsequently (Table 1). However, in PDMS-PmL eyes, OCT imaging demonstrated that retinal anatomy was well integrated and the films were placed successfully and maintained stably in the subretinal space of pigs after two-year transplantation (FIG. 6A; Table 1). It revealed intact photoreceptor /RPE layer with complete retinal attachment over and around the implant without edema or atrophy. The retina and RPE on both sides of the implant look unaffected. Around the PDMS-PmL subretinal implantation area, the retinal vasculature was preserved without signs of fibrosis or atrophy by color fundi photography at 2 year (FIG. 6B).Moreover, we executed a two-year follow-up with examination inspection every 3 months. The results of our survey demonstrated that no inflammatory signs in anterior chamber and vitreous body were detected (see Table 1). It is also observed that the results of the cornea/lens by slit-lamp examination were normal; and the results of the vitreous media by color fundi photography are clear for all pigs, either PDMS or PDMS-PmL groups.

TABLE 1
Schematic Overview of the morphologic changes

| | | | Implant/Retina | Implant/Retina |
|---|---|---|---|---|
| | Post- | IOP (mmHg) | features | features |
| | implantation | by | by Color fundi | by OCT |
| Group | month | Pneumotonogram | photography | examination |
| PDMS | 6 | 15.2 ± 2.1 | Implant in situ | Implant stable |
| (n = 4) | | | Retina well- | Photoreceptor layer |
| | | | looking | mild edema |
| | 12 | 14.3 ± 2.3 | Implant in situ | Implant stable |
| | | | Retina | Photoreceptor/RPE |
| | | | whitening/atrophy | layer partial |
| | | | | degenerated |
| | 24 | 15.6 ± 1.5 | Implant in situ | Implant stable |
| | | | Retina atrophy | Photoreceptor/RPE |
| | | | | layer partial loss |
| PDMS-PmL | 6 | 15.1 ± 1.7 | Implant in situ | Implant stable |
| (n = 6) | | | Retina well- | Photoreceptor/RPE |
| | | | looking | layer intact |
| | 12 | 15.7 ± 1.9 | Implant in situ | Implant stable |
| | | | Retina well- | Photoreceptor/RPE |
| | | | looking | layer intact |
| | 24 | 15.3 ± 1.8 | Implant in situ | Implant stable |
| | | | Retina well- | Photoreceptor/RPE |
| | | | looking | layer intact |

Neither did we found other ocular complications such as conjunctiva, cornea, anterior chamber, lens, high intraocular pressure, vitreous body, retinal break, retinal detachment, hyperocular pressure, and retinal hemorrhage in the eyes of these six subjects after PDMS-PmL implantation.

Importantly, after 2-year transplantation, the results of scotopic-ERG recordings revealed the retinal function to light response in PDMS-PmL transplanted eyes were no significantly different from that recorded in the before surgery eyes or control eyes (FIG. 6C). Collectively, these results confirmed the long-term biostability and biosafety of the PDMS-PmL in-vivo.

7. Comparison between PDMS-PmL Implant and PDMS Implant.

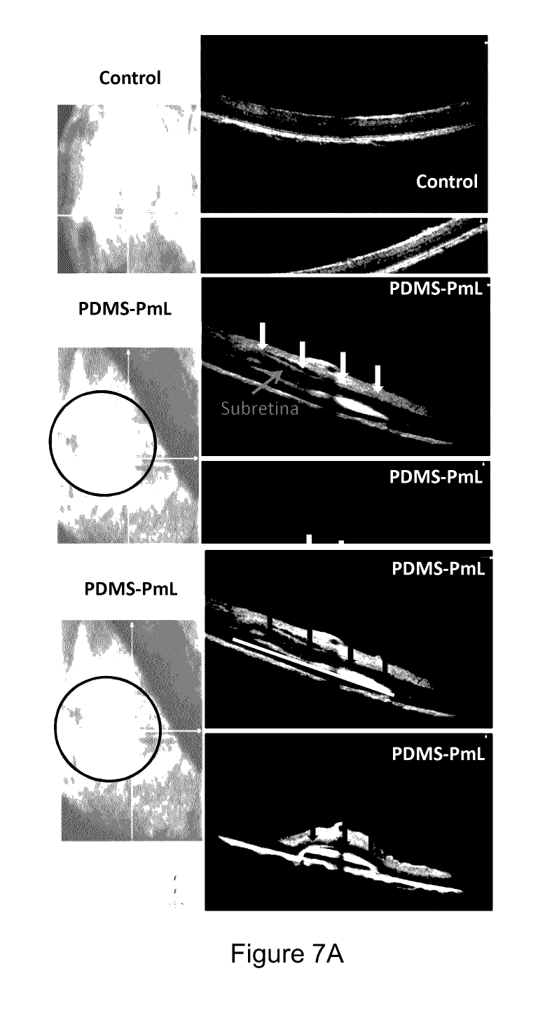

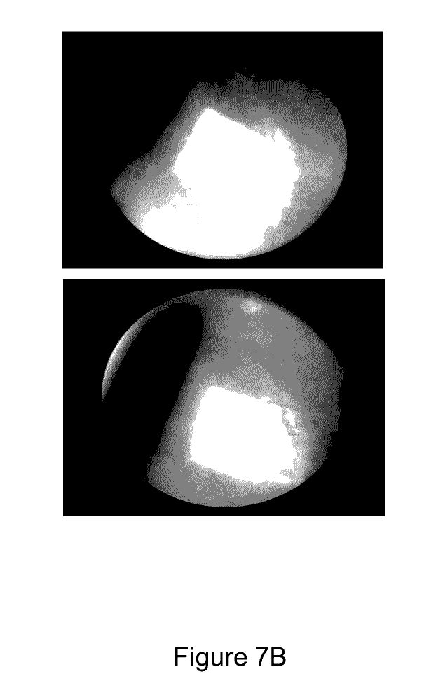

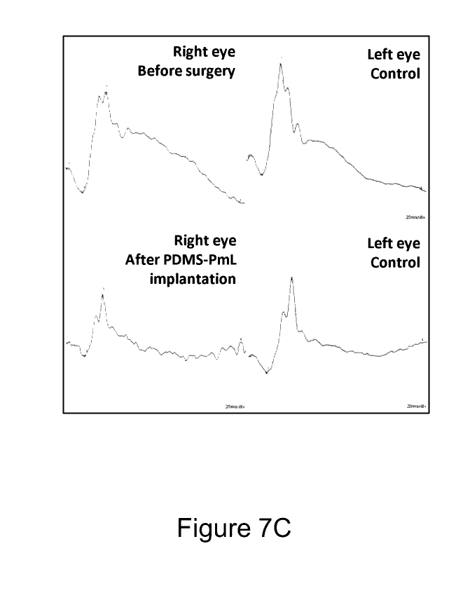

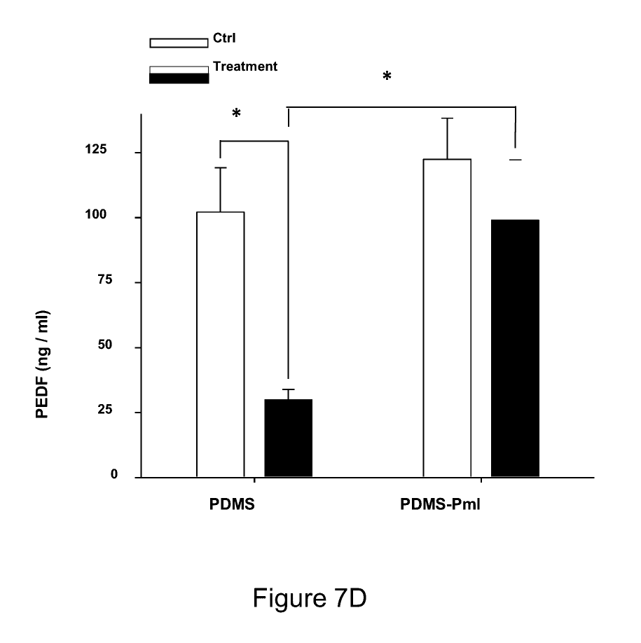

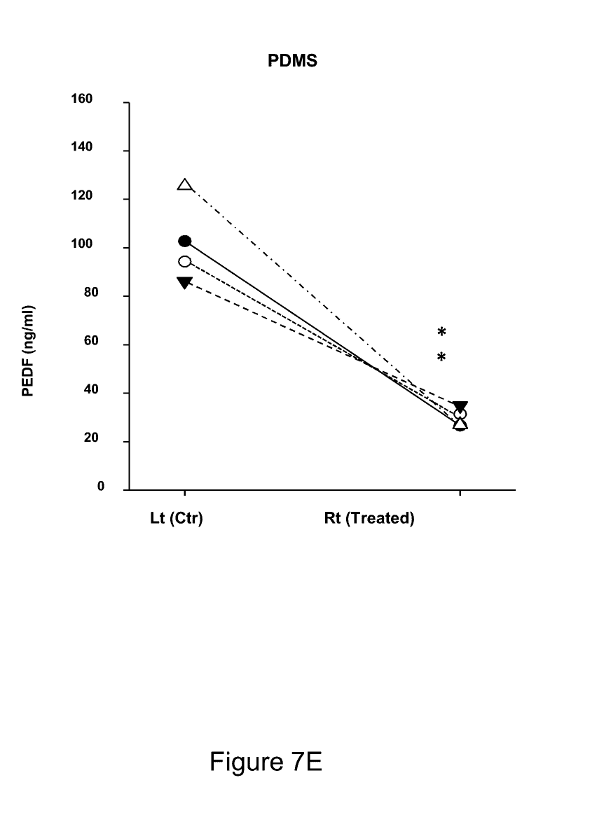

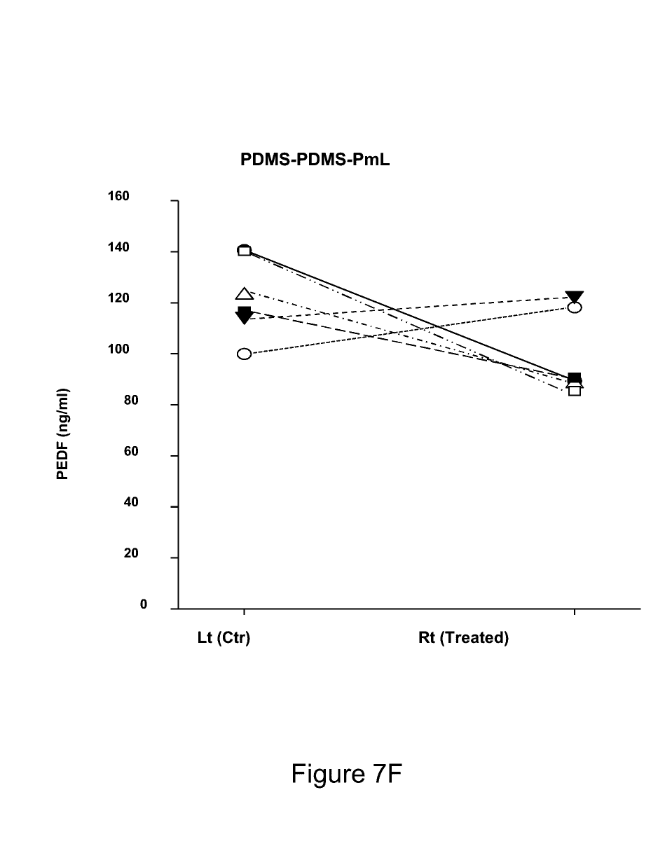

To evaluate the long-term biosafety and biostability of the PDMS implant and PDMS-PmL implant in vivo, the implantation of PDMS and PDMS-PmL were transplanted to the subretinal space around macular area in 4 and 6 porcine eyes, respectively. In PDMS-PmL eyes, OCT imaging demonstrated that retinal anatomy was well integrated and the films were placed successfully and maintained stably in the subretinal space of pigs after two-year transplantation. (see FIG. 7A) The retina and RPE on both sides of the implant look unaffected. Around the PDMS-PmL subretinal implantation area, the retinal vasculature was preserved without signs of fibrosis or atrophy by color fundi photography at 2 year (see FIG. 7B). After 2-year transplantation, the results of scotopic-ERG recordings revealed the retinal function to light response in PDMS-PmL transplanted eyes were no significantly different from that recorded in the before surgery eyes. (see FIG. 7C).

The PDMS-PmL transplantation up to 2 years maintained macula function and provide a good microenvironment for subreinal membranous scaffolds. We also isolated the macula area to detect the PEDF levels in PDMS and PDMS-PmL-transplanted eyes. Comparing to decreased levels of PEDF after PDMS transplantation, maintenance of trophic PEDF levels in PDMS-PmL eyes were observed to preserve retinal microenvironment in PDMS-PmL eyes at 2 year (FIG. 7D, FIG. 7E and FIG. 7F). Taken together, these data indicated that PDMS-PmL is able to sustain the physiological morphology and functions of polarized RPE monolayer, and demonstrated the potential application of PDMS-PmL transplantation for rescuing macular degeneration in vivo.

8. Long-Term Function of PDMS Implant and PDMS-PmL Implant

Retinal macula is located in the center of retina and responsible for central, high-resolution vision. However, how to measure the macular function of patients with retinal degeneration after stem cell transplantation or bionic retinal-implants is still an open question. Multi-focal ERG (mtERG) can provide an objective approach to analyze the local electrophysiological light-responses, including macular region, in AMD patients, as well as in large animals.

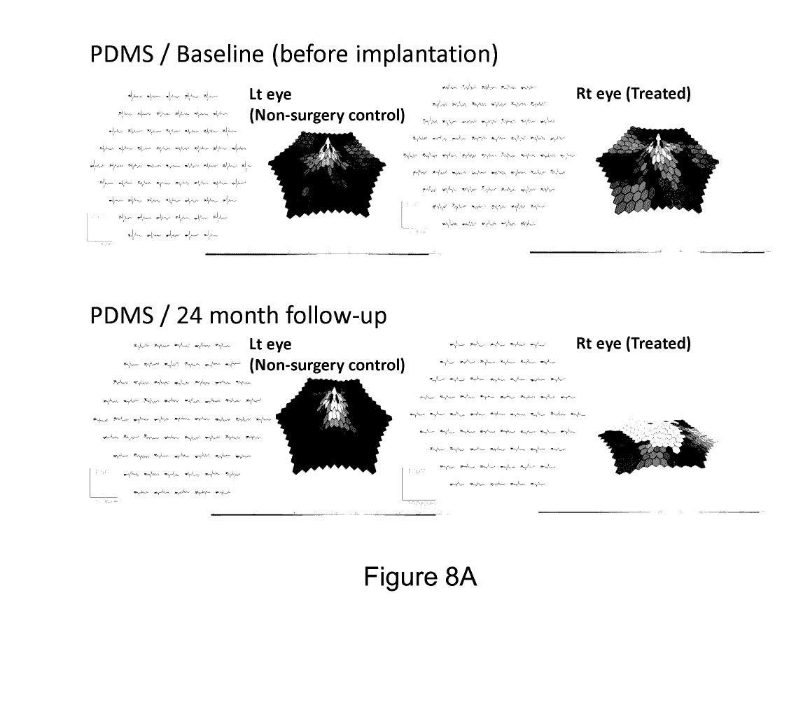

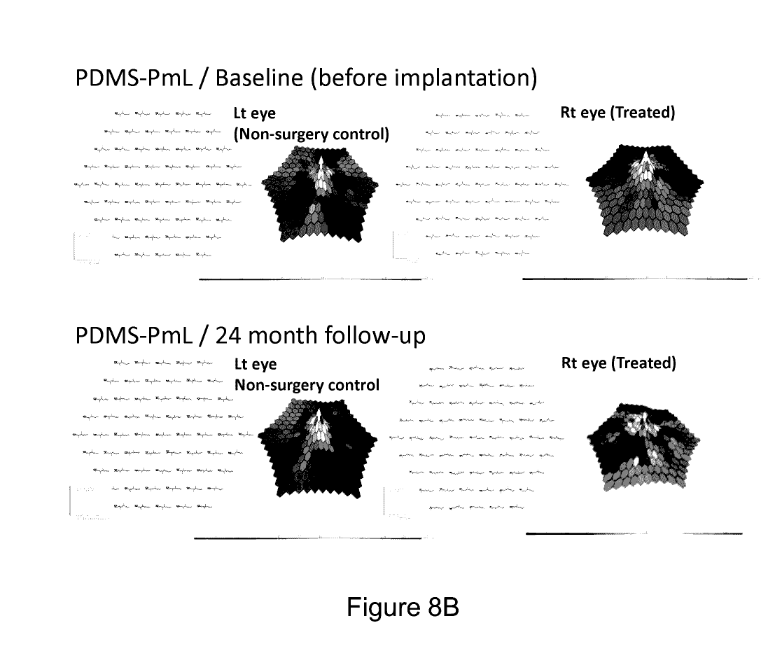

The PDMS implant and PDMS-PmL implant were transplanted in the subretinal space of transplanted porcine. At each time points, the right panels are 3D-topographical maps, and the right panels are trace array for the individual recordings of mtERG. Using mtERG as an platform to evaluate the macular function, right PDMS-transplanted eyes revealed partial depression of mtERG traces at 2 year, suggesting PDMS-related retinal injury (FIG. 8A).

However, mfERG signals in PDMS-PmL eyes were preserved generally at 2-year follow-up (FIG. 8B).

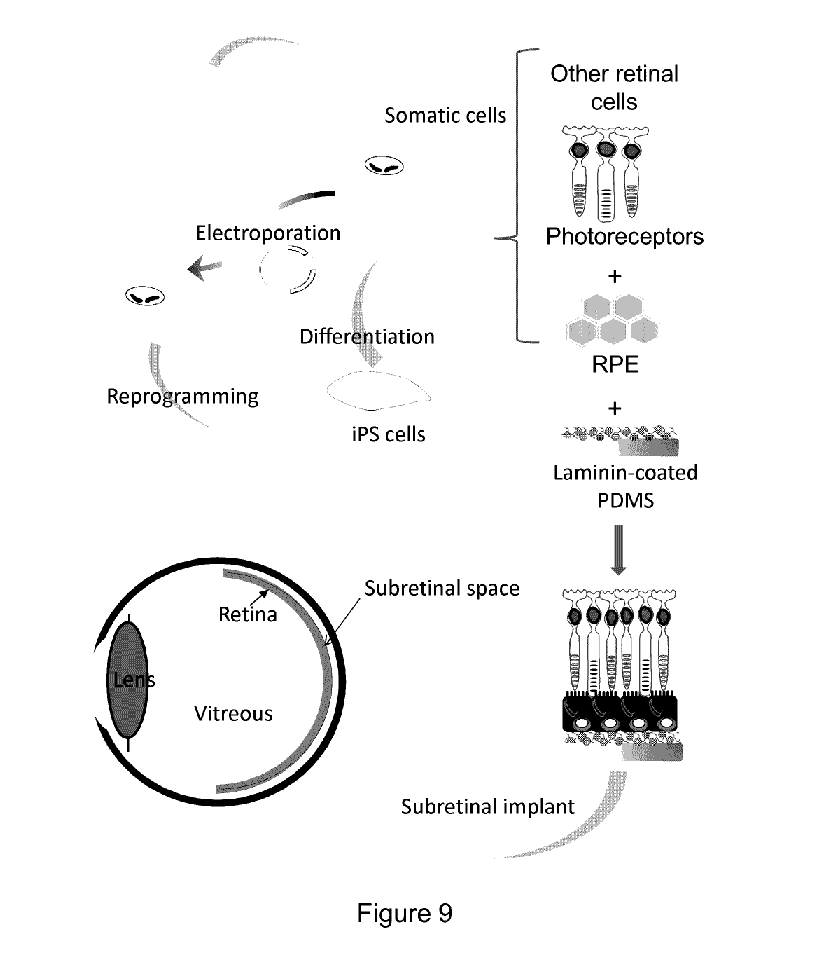

Given the above, it can be concluded that PDMS-PmL is able to sustain the physiological morphology and functions of polarized RPE monolayer, and demonstrated the in vivo effectiveness of dRPE/PDMS-PmL in increasing light response. As shown in FIG. 9, a potential application of dRPE/PDMS-PmL device in AMD patients can be developed.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

I/We claim:

1. A method for preparing a multilayered retinal cell implant comprises coating a substrate with laminin to obtain a laminin modified substrate and growing retinal cells derived from stem cells or induced pluripotent stem cells (iPSCs) on the laminin modified substrate, wherein the retinal cells as grown include multiple layers of retinal cells, and provides properties and efficacy facilitating retinal repair.

2. The method of claim 1, wherein the substrate comprises a sheet of a biocompatible polymeric compound.

3. The method of claim 2, wherein the biocompatible polymeric compound is selected from the group consisting of a polydimethylsiloxane (PDMS), poly(methyl methacrylate) (PMMA), Glycidoxypropyltrimethoxysilane (GPTES), aminopropyltriethoxysilane (APTES), Xylene and acetone. One example of the substrate used in the invention is polydimethylsiloxane (PDMS).

4. The method of claim 1, wherein the substrate comprises polydimethylsiloxane (PDMS).

5. The method of claim 1, wherein the substrate is subject to a surface modification with laminin by chemical and oxygen plasma treatment.

6. The method of claim 1, wherein the laminin modified substrate serves as a mimicking subretinal bruchs' basement.

7. The method of claim 1, wherein the laminin modified substrate facilitates the in vitro growth, phagocytosis, and Pigment epithelium-derived factor (PEDF) secretion of the RPE cells.

8. A multilayered retinal cell implant obtained by the method of claim 1.

9. The multilayered retinal cell implant of claim 8, which is obtained by the method of claim 4.

10. The multilayered retinal cell implant of claim 8, comprising multiple layers of various retinal cells.

11. The multilayered retinal cell implant of claim 9, comprising multiple layers of various retinal cells.

12. The multilayered retinal cell implant of claim 8, comprising retinal pigment epithelium (RPE) cells and photoreceptors.

13. The multilayered retinal cell implant of claim 9, comprising retinal pigment epithelium (RPE) cells and photoreceptors.

14. The multilayered retinal cell implant of claim 8, which maintains pigment epithelium-derived factor (PEDF) secretion.

15. The multilayered retinal cell implant of claim 9, which maintains pigment epithelium-derived factor (PEDF) secretion.

16. A method for repairing a retinal defect within an eye of a subject in need thereof, comprising transplanting on the retinal defect the multilayered retinal cell implant obtained by the method of claim 1.

17. The method of claim 16, wherein the multilayered retinal cell implant is obtained by the method of claim 4.
